(19) United States
(12) Patent Application Publication
(10) Pub. No.: US 2010/0075020 A1
(43) Pub. Date: Mar. 25, 2010

(54) METHODS FOR COATING FILAMENTS

(75) Inventor: Jon Reinprecht, Watertown, CT (US)

Correspondence Address: Tyco Healthcare Group LP, 60 MIDDLETOWN AVENUE, NORTH HAVEN, CT, 06473, US

(73) Assignee: Tyco Healthcare Group LP

(21) Appl. No.: 12/507,453

(22) Filed: Jul. 22, 2009

Related U.S. Application Data
(60) Provisional application No. 61/099,957, filed on Sep. 25, 2008.

(51) Int. Cl. A61F 13/02

(52) U.S. Cl. 427/2.31

(57) ABSTRACT

A method for applying a coating composition to filaments whereby filaments are wrapped about an outer perimeter of a drum. The filament-wrapped drum is placed within a dip tank, the dip tank including a coating composition, and a least a portion of the filaments are thereby coated.

### CROSS REFERENCE TO RELATED APPLICATIONS

This application claims priority to, and the benefit of, U.S. Provisional Patent Application Ser. No. 61/099,957 filed on Sep. 25, 2008, the entire content of which is incorporated herein by reference.

### TECHNICAL FIELD

The present disclosure relates to methods for coating filaments, and more specifically to methods for coating sutures.

### BACKGROUND OF RELATED ART

Techniques for coating filaments, e.g. sutures, are known. Coatings may benefit sutures by improving the strength or knot tie-down characteristics, or adding color to sutures. Coatings may also be formulated to provide therapeutic benefits to the tissue as a drug carrier.

Generally, coatings are applied by dipping, bushing, wiping, spraying, drip coating, or by use of a coating/filling head. These coating processes involve passing a suture line into or through a coating composition. In conventional suture coating techniques, the suture must be unwound in order to pass the suture line through the coating. Improvements in the field of coating filaments are desired.

### SUMMARY

A method of coating a filament is disclosed herein, the method including the steps of providing a filament wrapped about an outer perimeter of a drum; and, dipping at least a portion of the outer perimeter of the drum into a dip tank containing a first coating composition thereby coating a portion of the filament. The filament may have a length defined by the outer perimeter of the drum. Furthermore, the filament may have a length-to-outer perimeter ratio ranging from about 0.5:1 to about 5000:1, and in some embodiments, from about 1:1 to about 3000:1. The filament may comprise a monofilament or a multifilament suture.

The coated portion of the filament may represent about 25% to about 75% of the length of the filament, and in some embodiments, about 50% of the length of the filament. The first coating composition may provide a color pattern along a longitudinal portion of the filament. In some embodiments, the first coating composition is selected from the group consisting of dyes, pigments, colorants, adhesives, bioactive agents, lubricants and combinations thereof. Additionally the method may include the step of applying a second coating composition to the filament. The second coating composition may be selected from the group consisting of dyes, pigments, colorants, adhesives, bioactive agents, lubricants and combinations thereof. Furthermore, the second coating composition may be applied to an uncoated portion of the filament.

Additionally, the method of coating a filament includes a filament wrapped thereabout a drum. Drums of the present disclosure may have a geometrical configuration selected from the group consisting of triangular, cylindrical, pyramidal, square, rectangular, octagonal, hexagonal, pentagonal and polygonal. In certain preferred embodiments, the drum is cylindrical.

In some embodiments, the dip tank includes a level of the coating composition at least equal to a midpoint of the drum.

The disclosed method may also comprise the step of releasing the filament from the drum.

A method of forming a bi-colored filament is also disclosed, the method comprises the steps of providing a filament wrapped about an outer perimeter of a drum; and, dipping at least a portion of the outer perimeter of the drum into a dip tank containing a liquid colorant thereby creating a color pattern on a portion of the filament. The color pattern may be applied to a longitudinal portion of the filament.

A system for coating filaments is also disclosed herein, the system including a filament wrapped about an outer portion of a drum; and a dip tank containing a coating composition, wherein the dip tank is configured to receive the drum therein to at least a mid-point of the drum, thereby coating about 50% of the filament with the coating composition.

### BRIEF DESCRIPTION OF DRAWINGS

The illustrative embodiments described herein will become more readily apparent from the following description, reference being made to the accompanying drawings in which:

FIG. 1A is a perspective view of a dip system in accordance with the present disclosure;

FIG. 1B is a perspective view of a drum included in the dip system of FIG. 1;

FIG. 2A is plan view of a second embodiment of a drum in accordance with a dipping method of the present disclosure;

FIG. 2B is a plan view of an alternate embodiment of a drum in accordance with the dipping method of the present disclosure;

FIG. 3A is a perspective view of yet another embodiment of a drum in accordance with the dipping method of the present disclosure;

FIG. 3B illustrates a coated suture made in accordance with the drum of FIG. 3A; and,

FIG. 4 is a perspective view of another embodiment of a drum further including a mask in accordance with the dipping method of the present disclosure.

### DETAILED DESCRIPTION OF EMBODIMENTS

The present disclosure is directed to a method of coating a filament. At least one length of a filament is wrapped about a drum and the drum is subsequently dipped in a dip tank filled with a coating composition. The method of coating a filament may be used to coat medical devices including, but not limited to, sutures, tapes, and mesh. The term “filament” as described herein means a continuous strand that has an elongate body portion. In some embodiments, several filaments may be knitted, woven, fused or braided together, creating a multifilament or “fiber.”

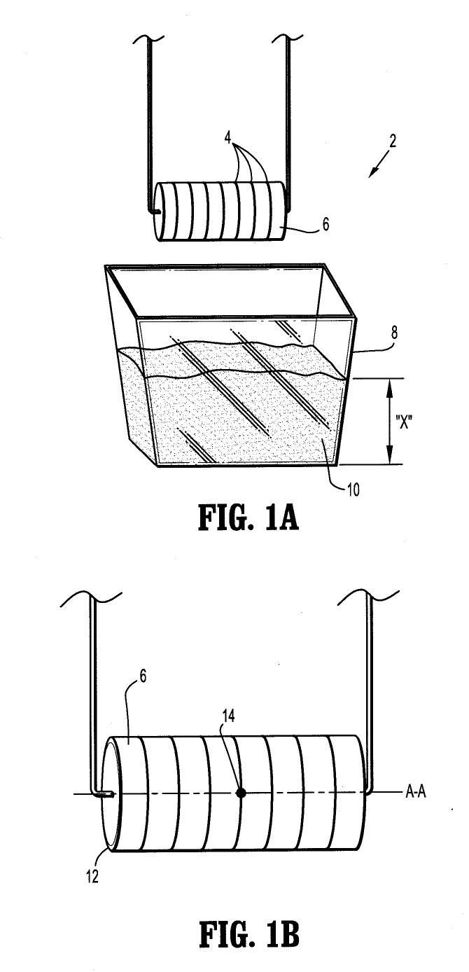

FIG. 1 illustrates a dipping system 2 for coating filaments 4 wrapped about a drum 6. The dipping system 2 includes a dip tank 8 filled with a coating composition 10. Any reservoir or tank adapted to hold a volume of a coating composition 10 (coating solution) capable of submerging or contacting filaments 4 wound about a drum 6 may be utilized. Tank 8 may have various shapes such as rectangular or cylindrical, or tank 8 may be shaped to compliment the geometry of the drum 6. Tank 8 may be made of any suitable material which is capable of containing a coating composition 10 or which is generally non-reactive with the coating composition 10.

The drum 6 may have a desired filament length wrapped thereabout. In some embodiments, the filament 4 has a length defined by the outer perimeter 12 of the drum 6. The outer perimeter 12 is illustrated at one end of the drum, it being understood that the outer perimeter is uniform across the length of a cylindrical drum 6. The term “length-to-outer perimeter ratio” will be used herein and defines the ratio of the length of the filament 4 to the outer perimeter 12 of the drum 6. In some embodiments, the filament 4 has a length-to-outer perimeter ratio ranging from about 0.5:1 to about 5000:1, more specifically, from about 1:1 to about 3000:1. For example, if the length-to-outer perimeter ratio is 1:1, the length of the filament 4 is the same length as the length of the outer perimeter 12 of the drum 6. For a cylindrical drum, the length of the outer perimeter is equal to the length of the circumference. In another example, a cylindrical drum 6 with a 2:1 length-to-outer perimeter ratio, one filament is wrapped about the cylindrical drum two times. The length of the filament is twice the length of the circumference (outer perimeter) of the drum.

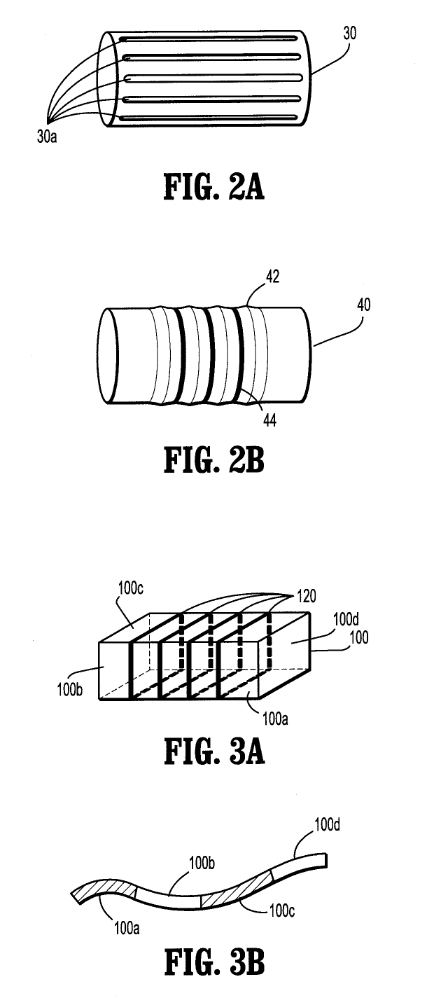

Filaments are wrapped about a drum prior to contacting the coating composition. One skilled in the art may envision several methods for wrapping filaments about drums. FIGS. 1A and 1B illustrate a drum 6 with several filaments 4 wrapped thereabout. The drum 6 is cylindrical in geometry, having a smooth circumferential surface. In some embodiments, the drum may be made of stainless steel or stainless steel covered with a silicon rubber skin. In other embodiments, the drum is comprised of high density polyethylene with steel end plates. High density polyethylene may be preferable when coating filaments with specific agents. For example, cyanoacrylate does not adhere to high density polyethylene, which allows excess coating (cyanoacrylate) to run off the drum and back into a dip tank. It should be noted that the drum 6 may be constructed of any suitable material which is compatible with the filaments 4 and the coating composition 10 utilized in the system 2. The drum 6 may be of solid or hollow construction. Yet, in other embodiments, the drum 30 may be perforated (FIG. 2A). The perforations 30a allow a coating to coat the filaments which are closest to the drum center. The perforations 30a may be of any size and shape and may extend along the length or any portion of the drum 30. Alternatively, FIG. 2B illustrates a drum 40 including ridges or ribs 42 along the outside perimeter of the drum 40 to keep the filaments 44 separate during the coating process.

Once the filaments 4 are wrapped about a drum 6, the drum 6 is contacted with the coating solution 10. The drum 6 (including filaments) is placed into a dip tank 8 containing a first coating composition 10, and at least a portion of the outer perimeter of the drum is dipped into the coating composition, thereby coating a portion of the filaments. In some embodiments, the drum 6 may be placed into the dip tank 8 using manual techniques or various mechanical devices which are within the purview of those skilled in the art. Furthermore, the dip tank 8 includes a level of a coating composition 10 which, in some embodiments, is at least equal to a midpoint 14 of the drum 6. The midpoint 14 of the drum 6 is a point taken to be the central most point 14 along the centerline A-A of the drum 6. As shown, the length-to-outer perimeter ratio is about 1:1 and a coating composition is at a level X in the dip tank 8. The filament-wrapped drum 6 is placed in the dip tank 8 with the coating composition for a specific period of time. In some embodiments, the drum 6 may be contacted with the coating composition 10 from about 30 seconds to about 2 hours or more, depending on the filament material and the compositional make-up of the coating. Optionally, once a first portion of the filament 4 is coated, the drum 6 may be rotated and lowered into the dip tank 8 a second time (optionally with a different coating), coating a second portion of the filament 4. The coating process may be repeated until the desired coating level and coating materials have been applied to the filament 4.

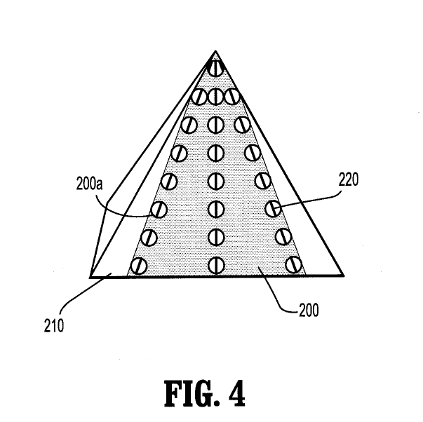

As illustrated in FIG. 1, filaments 4 are wrapped about a cylindrical drum 6. Drums may have other geometrical configurations including polygonal shapes such as triangular, pyramidal, rectangular, hexagonal, octagonal, and pentagonal. Wrapping filaments about drums with various geometries may be useful when more than one coating is applied or when a coating is applied to various lengths. For example, when a pyramidal drum (such as FIG. 4) is used for coating a suture, three different portions of a suture may be coated with three different coatings. More specifically, a first portion of a suture may be coated with a cyanoacrylate, providing stiffness to a first portion of the suture, e.g., for needle attachment. A second coating may be applied to a middle portion of the suture, to increase lubricity and knot rundown. A third coating may be applied to a third portion of the suture to obtain preferable handling characteristics. As another example, a rectangular drum may be used (as shown in FIG. 3A) to obtain a color patterned filament shown in FIG. 3B. The rectangular drum 100 includes several filaments 120 wrapped thereabout, wherein the length-to-outer perimeter is about 1:1. The drum 100 includes four sides (100a, 100b, 100c, 100d) which may be dipped into a dip tank. Filament portions 120a and 120c are in contact with sides 100a and 100c. Drum sides 100a and 100c are contacted with a colored coating composition (drum may rotate between coating applications), coloring portions 120a and 120c of the filaments 120. When the filaments 120 are removed from the drum 100, portions 120a and 120c of the filaments 120 include a solid color pattern (FIG. 4B).

Additionally, filaments may be wrapped about a drum in various patterns including a single layer of filaments as illustrated in FIG. 1. The filaments 4 may also be wrapped about the drum in a cross-wise pattern (not shown) or conversely, the filaments may be wrapped about the drum, such that the filaments are stacked on top of one another (not shown). The filaments may be wrapped about the drum in any configuration depending on the surface area exposure desired or the length of filament and/or coating time desired for contacting coating compositions.

Various coating patterns can be applied to the surface of filaments. Coating patterns as defined herein may include any coating patterns, solid, checkered, striped, random, or otherwise, such that the coating is present on a specific area of the filament. Coatings may be applied to a longitudinal portion of the filaments to create a coating pattern. Patterned coatings may also be created by masking or covering specific portions of the filament so as to enable the coating composition to be applied to unmasked portions of the filament, as shown in FIG. 4. A mask 200 is shown covering portions of filaments 220, which are wrapped about a drum 210. The mask 200 includes perforations or holes 200a which when placed in a dip tank filled with a coating composition, enable the coating composition to contact the filaments 220 and coat the unmasked or exposed filaments portions. It should be understood that masks may allow a variety of coating patterns, including solid patterns, along the filament surface, and mask patterns are not limited to those illustrated herein. In general, the coated portion of a single filament represents from about 25% to about 75% of the length of the filament.

Once the coating has been applied to the filament, the filament-wrapped drum is separated from the coating composition. The drum may either be removed from the dip tank, or conversely, the coating composition may be drained from the dip tank. Optionally, once the first coating composition is drained from the dip tank, a second coating composition may fill the dip tank for a second coating and the drum may be lowered into the second coating composition. In some embodiments, the drum may be rotated between coating applications, while in other embodiments the drum may not be rotated between coatings. Once the filaments are dry, they are cut and released from the drum. It should be noted that filaments may be cut to various lengths. One skilled in the art may envision various methods of releasing the filaments from the drum such as thermal energy or lasers.

Coating compositions may be applied to any filaments such as sutures, yarns, tapes, or mesh. The coating compositions may be in the form of a solution, dispersion, emulsion or any other homogeneous or heterogeneous mixture.

In one embodiment, coating compositions may include color additives, dyes or pigments. Filaments may be coated with a coating composition including color additives such as those approved by the Food and Drug Administration (FDA) for various non-bioabsorbable and bioabsorbable sutures, including, but not limited to, chromium-colbalt-aluminum oxide, ferric ammonium citrate, pyrogallol, logwood extract, D&C Blue No. 9, D&C Green No. 5, [phthalocynaninato(2-)copper], FD&C Blue No. 2, D&C Blue No. 6, D&C Green No. 6, D&C Red No. 17, and D&C Violet No. 2.

Cyanoacrylates may also be used as a coating composition. Suitable cyanoacrylates include materials derived from methyl cyanoacrylate, ethyl cyanoacrylate, butyl cyanoacrylate, octyl cyanoacrylate, isobutyl cyanoacrylate and methoxypropyl cyanoacrylate and combinations thereof and the like.

Additionally, bioactive agents may be incorporated into coating composition such as antimicrobial agents, anti-virals, anti-fungals, and the like. Antimicrobial agents as used herein is defined by an agent which by itself or through assisting the body (immune system) helps the body destroy or resist microorganisms which may be pathogenic (disease causing). The term “antimicrobial agent” includes antibiotics, quorum sensing blockers, surfactants, metal ions, antimicrobial proteins and peptides, antimicrobial polysaccharides, antiseptics, disinfectants, anti-virals, anti-fungals, quorum sensing blockers, and combinations thereof.

Other suitable bioactive agents which may be used include radiopaque markers, preservatives, protein and peptide preparations, protein therapeutics, polysaccharides such as hyaluronic acid, lectins, lipids, probiotics, angiogenic agents, anti-thrombotics, anti-clotting agents, clotting agents, analgesics, anesthetics, wound repair agents, chemotherapeutics, biologics, anti-inflammatory agents, anti-proliferatives, diagnostic agents, antipyretic, antiphlogistic and analgesic agents, vasodilators, antihypertensive and antiarrhythmic agents, hypotensive agents, antitussive agents, antineoplastics, local anesthetics, hormone preparations, antiasthmatic and antiallergic agents, antihistaminics, anticoagulants, antispasmodics, cerebral circulation and metabolism improvers, antidepressant and antianxiety agents, vitamin D preparations, hypoglycemic agents, antiulcer agents, hypnotics, antibiotics, antifungal agents, lubricants, sedative agents, bronchodilator agents, antiviral agents, dysuric agents, brominated or halogenated furanones, and the like. In embodiments, polymer drugs, i.e., polymeric forms of such compounds for example, polymeric antibiotics, polymeric antiseptics, polymeric chemotherapeutics, polymeric anti-proliferatives, polymeric antiseptics, polymeric non-steroidal anti-inflammatory drugs (NSAIDS), and the like may be utilized and combinations thereof.

In certain embodiments, coating compositions of the present disclosure may contain suitable medicinal agents such as viruses and cells, peptides, polypeptides and proteins, analogs, muteins, and active fragments thereof, such as immunoglobulins, antibodies (monoclonal and polyclonal), cytokines (e.g. lymphokines, monokines, chemokines), blood clotting factors, hemopoietic factors, interleukins (IL-2, IL-3, IL-4, IL-6), interferons (β-IFN, α-IFN and γ-IFN), erythropoietin, nucleases, tumor necrosis factor, colony stimulating factors (e.g., GCSF, GM-CSF, MCSF), insulin, anti-tumor agents and tumor suppressors, blood proteins, gonadotropins (e.g., FSH, LH, CG, etc.) hormones and hormone analogs (e.g., growth hormone), vaccines (e.g., tumoral, bacterial and viral antigens), somatostatin, antigens, blood coagulation factors, growth factors, protein inhibitors, protein antagonists, and protein agonists, nucleic acids, such as antisense molecules, DNA, RNA, oligonucleotides, polynucleotides and ribozymes and combinations thereof.

Coating compositions may be made within the purview of those skilled in the art including mixing, stirring, phase separation and the like. Furthermore, coating compositions may also include polar and non-polar solvents. Suitable solvents are within the purview of those skilled in the art.

Filaments of the present disclosure may be non-absorbable or absorbable. It should be understood that filaments and combinations of multifilaments can be made from different materials (e.g. natural and synthetic, or bioabsorbable and non-bioabsorbable materials).

Filaments may comprise synthetic absorbable materials including polymers such as those made from lactide, glycolide, caprolactone, valerolactone, carbonates (e.g., trimethylene carbonate, tetramethylene carbonate, and the like), dioxanones (e.g., 1,4-dioxanone) δ-valerolactone, 1,dioxepanones (e.g., 1,4-dioxepan-2-one and 1,5-dioxepan-2-one), ethylene glycol, ethylene oxide, esteramides, γ-hydroxyvalerate, β-hydroxypropionate, alpha-hydroxy acid, hydroxybuterates, orthoesters, hydroxy alkanoates, tyrosine carbonates, polyimide carbonates, polyimino carbonates such as poly (bisphenol A-iminocarbonate) and poly (hydroquinone-iminocarbonate), and polymer drugs (e.g., polydiflunisol, polyaspirin, and protein therapeutics) and copolymers and combinations thereof. Suitable natural absorbable polymers include collagen, cellulose and gut. In embodiments, glycolide and lactide based polyesters, including copolymers of lactide and glycolide may be used.

Suitable non-absorbable materials which may be used to form filaments include non-absorbable natural materials such as cotton, silk, and rubber. Suitable non-absorbable synthetic materials include monomers and polymers derived from materials such as nylons, polyolefins such as polypropylene and polyethylene, ultra high molecular weight polyethylene (UHMWPE), polyamides, polyesters such as poly ethylene terepththalate (PET), polyaryletherketone, polyvinylidene difluoride (PVDF), acrylic, polyamides, aramids, fluropolymers, polybutesters, silicones, and polymer blends, copolymers thereof and combinations with degradable polymers. In embodiments, polypropylene can be utilized to form the suture. The polypropylene can be isotactic polypropylene or a mixture of isotactic and syndiotactic or atactic polypropylene. Additionally, non-absorbable synthetic and natural polymers and monomers may be combined with each other and may also be combined with various absorbable polymers and monomers to create fibers.

In some embodiments, filaments may comprise shape memory polymers. Suitable polymers used to prepare hard and soft segments of shape memory polymers include polycaprolactone, dioxanone, lactide, glycolide, polyacrylates, polyamides, polysiloxanes, polyurethanes, polyether amides, polyurethane/ureas, polyether esters, and urethane/butadiene copolymers and combinations thereof. Methods for making the described filaments from these suitable bioabsorbable and non-bioabsorbable materials are within the purview of those skilled in the art (e.g. extrusion and molding).

It should be noted that filaments may be combined to create a multifilament medical device. In some embodiments, filaments may be combined prior to a coating application or coating step, in other embodiments, filaments may be combined after a coating application. Several filaments can be combined in any manner for use in the present disclosure. The plurality of filaments may be combined using any technique within the purview of one skilled in the art such as commingling, twisting, braiding, weaving, entangling, and knitting. For example, a plurality of filaments may simply be combined to form a yarn. As another example, a plurality of filaments may be braided. As yet another example, a plurality of filaments may be combined to form a yarn and then those multifilament yarns may be braided. Those skilled in the art reading this disclosure will envision other ways in which filaments may be combined. Filaments may also be combined to produce a non-woven multifilament fiber.

What is claimed is:

1. A method of coating a filament, the method comprising the steps of:
providing a filament wrapped about an outer perimeter of a drum; and, dipping at least a portion of the outer perimeter of the drum into a dip tank containing a first coating composition thereby coating a portion of the filament.

2. The method of claim 1, wherein the filament has a length defined by the outer perimeter of the drum.

3. The method of claim 2, wherein the filament has a length-to-outer perimeter ratio ranging from about 0.5:1 to about 5000:1.

4. The method of claim 2, wherein the filament has a length-to-outer perimeter ratio ranging from about 1:1 to about 3000:1.

5. The method of claim 1, wherein the coated portion of the filament represents about 25% to about 75% of the length of the filament.

6. The method of claim 1, wherein the coated portion of the filament represents about 50% of the length of the filament.

7. The method of claim 1 wherein the filament is a monofilament suture.

8. The method of claim 1 wherein the filament is a multifilament suture.

9. The method of claim 1, wherein the first coating composition provides a color pattern along a longitudinal portion of the filament.

10. The method of claim 1, wherein the first coating composition is selected from the group consisting of dyes, pigments, colorants, adhesives, bioactive agents, lubricants and combinations thereof.

11. The method of claim 1, wherein the drum is has a geometrical configuration selected from the group consisting of triangular, cylindrical, pyramidal, square, rectangular, octagonal, hexagonal, pentagonal and polygonal.

12. The method of claim 1, wherein the drum is cylindrical.

13. The method of claim 1, wherein the dip tank includes a level of the coating composition at least equal to a midpoint of the drum.

14. The method of claim 1, further comprising the step of applying a second coating composition to the filament.

15. The method of claim 14, wherein the second coating composition is selected from the group consisting of dyes, pigments, colorants, adhesives, bioactive agents, lubricants and combinations thereof.

16. The method of claim 14, wherein the second coating composition is applied to an uncoated portion of the filament.

17. The method of claim 1, further comprising the step of releasing the filament from the drum.

18. A method of forming a bi-colored filament, the method comprising the steps of:
providing a filament wrapped about an outer perimeter of a drum; and, dipping at least a portion of the outer perimeter of the drum into a dip tank containing a liquid colorant thereby creating a color pattern on a portion of the filament.

19. The method of claim 18, wherein the color pattern is applied to a longitudinal portion of the filament.

20. A system for coating filaments comprising:
a filament wrapped about an outer portion of a drum; and
a dip tank containing a coating composition, wherein the dip tank is configured to receive the drum therein to at least a mid-point of the drum, thereby coating about 50% of the filament with the coating composition.
